(19) United States
(12) Patent Application Publication
(10) Pub. No.: US 2023/0062811 A1
(43) Pub. Date: Mar. 2, 2023

(54) SYSTEMS AND METHODS FOR PROCESSING ELECTRONIC IMAGES IN FORENSIC PATHOLOGY

(71) Applicant: PAIGE.AI, Inc., New York, NY (US)

(72) Inventors: Jeremy Daniel KUNZ, New York, NY (US); Christopher KANAN, Pittsford, NY (US); Ran GODRICH, New York, NY (US); Patricia RACITI, New York, NY (US); Mindy FERSEL, Englewood, NJ (US)

(21) Appl. No.: 17/877,319

(22) Filed: Jul. 29, 2022

Related U.S. Application Data
(60) Provisional application No. 63/236,330, filed on Aug. 24, 2021.

(51) Int. Cl. G16H 50/20; G16H 30/40; G16H 15/00

(52) U.S. Cl. CPC G16H 50/20; G16H 30/40; G16H 15/00

(57) ABSTRACT

A computer-implemented method for processing electronic medical images, the method including receiving images of at least one pathology specimen, the pathology specimen being associated with a patient. The system may determine, using a machine learning system and based on the electronic medical images, at least one contributing cause of death. The system may provide at least contributing cause of death.

### RELATED APPLICATION(S)

This application claims priority to U.S. Provisional Application No. 63/236,330 filed Aug. 24, 2021, the entire disclosure of which is hereby incorporated herein by reference in its entirety.

### FIELD OF THE DISCLOSURE

Various embodiments of the present disclosure pertain, generally, to image processing methods. More specifically, particular embodiments of the present disclosure relate to systems and methods predicting causes of death including artificial intelligence system for inferring the likely causes of death, based on medical images.

### BACKGROUND

Identifying a cause of death by necropsy/autopsy can be a time consuming, costly, invasive and imprecise process that, at times, requires expert knowledge that may not result in a cause of death determination. These drawbacks may lead to unresolved cases for law enforcement, extensive forensic examination, and a lack of emotional closure for deceased individuals' loved ones. As a result, the identification process might not be undertaken because of the potential drawbacks.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the present disclosure, systems and methods are disclosed for processing electronic medical images, comprising: receiving images of at least one pathology specimen, the pathology specimen being associated with a patient; determining, using a machine learning system and based on the electronic medical images, at least one contributing cause of death; providing at least contributing cause of death.

A system for processing electronic medical images, the system including: at least one memory storing instructions; and at least one processor configured to execute the instructions to perform operations including: receiving images of at least one pathology specimen, the pathology specimen being associated with a patient; determining, using a machine learning system and based on the electronic medical images, at least one contributing cause of death; providing at least contributing cause of death.

A non-transitory computer-readable medium storing instructions that, when executed by a processor, perform operations processing electronic medical images, the operations including: receiving images of at least one pathology specimen, the pathology specimen being associated with a patient; determining, using a machine learning system and based on the electronic medical images, at least one contributing cause of death; providing at least contributing cause of death.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates an exemplary block diagram of a system and network for processing images, for example image matching, according to techniques presented herein.

FIG. 1B illustrates an exemplary block diagram of a tissue viewing platform according to techniques presented herein.

FIG. 1C illustrates an exemplary block diagram of a slide analysis tool, according to techniques presented herein.

FIG. 2 illustrates a process for determining a forensic analysis, according to techniques presented herein.

FIG. 3A is a flowchart illustrating an example method for training an algorithm for determining image region detection, according to techniques presented herein.

FIG. 3B is a flowchart illustrating methods for image region detection, according to one or more exemplary embodiments herein.

FIG. 4A is a flowchart illustrating an example method for training an algorithm for determining one or more causes of death, according to techniques presented herein.

FIG. 4B is a flowchart illustrating exemplary methods for determining one or more cause of death, according to one or more exemplary embodiments herein.

FIG. 5A is a flowchart illustrating an example method for training an algorithm to recognize a cardiovascular disease cause of death, according to techniques presented herein.

FIG. 5B is a flowchart illustrating exemplary methods for determining causes of cardiovascular disease, according to one or more exemplary embodiments herein.

FIG. 6 is a flowchart illustrating an exemplary method for determining liver toxin analysis, according to one or more exemplary embodiments herein.

FIG. 7 is a flowchart illustrating an exemplary method for detecting infection, according to one or more exemplary embodiments herein.

FIG. 8 is a flowchart illustrating exemplary methods for determining one or more autopsy report fields, according to one or more exemplary embodiments herein.

FIG. 9 is a flowchart illustrating exemplary methods for predicting a cause of a miscarriage, according to one or more exemplary embodiments herein.

FIG. 10 is a flowchart illustrating exemplary methods for determining cause of death, according to one or more exemplary embodiments herein.

FIG. 11 depicts an example of a computing device that may execute techniques presented herein, according to one or more embodiments.

### DESCRIPTION OF THE EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Techniques presented herein describe determining a cause of death using computer vision and/or machine learning and/or predicting the fields on an autopsy report, cardiac arrhythmogenic genes, contributing factors of cardiovascular disease, liver toxins, and/or cause of miscarriage.

Techniques presented herein may relate to using scans of digital medical images and additional information for an individual followed by using image processing techniques and/or machine learning to determine a cause of death.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Deep learning techniques may also be employed. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Autopsies may involve the gross dissection and examination of a deceased human body paired with histologic examination of tissues from the deceased in order to establish manner and cause of death. Microscopic analysis of the representative cell and tissue samples taken from the major internal organs may be undertaken by a pathologist in order to generate a report of findings and determine the final cause of death. Even after an autopsy is undertaken, a definitive cause of death might not be discovered and may be purely hypothesized.

Traditional autopsies may be time-consuming, resource-intensive, and expensive procedures that may be offered to a decedent's family or for legal investigations to determine cause of death. Autopsies may also be highly invasive procedures that involve opening chest and abdominal cavities as well as a skull to gain access to the appropriate organs for gross examination and histologic sampling. Autopsies can yield valuable genetic information that can contribute not only to understanding how and why the decedent died, but also may inform possible carrier status of deleterious genetic mutations that can inform family members of their own health risks.

Solutions provided herein more definitively and accurately establish the cause of death in a less invasive manner (e.g., in comparison to traditional autopsies) and provide more universal assessment of genetic information (e.g., for the potential benefit of family members).

According to certain aspects of the disclosure, methods and systems are disclosed for providing a system/process configured for determining one or more fields in the autopsy report, toxin report, cardiovascular report, or for inferring one or more causes of death. Certain embodiments of the disclosed subject matter are directed to applying artificial intelligence (AI)/machine learning (ML) models to whole slide images (WSIs) of tissue from deceased humans and animals to train the AI/ML to determine mechanisms and/or causes of death, as will be discussed in greater detail below.

As will be discussed in more detail below, in various embodiments, systems and methods are described for utilizing image processing techniques and/or machine learning techniques to determine one or more fields in the autopsy report, toxin report, cardiovascular report, or to infer one or more causes of death.

Further, in various embodiments, systems and methods are described for using various machine learning techniques in order to determine one or more fields in the autopsy report, toxin report, cardiovascular report, or to infer one or more causes of death. By training one or more autopsy/pathology inference AI models, e.g., via supervised, semi-supervised learning, or supervised learning to learn how to determine one or more fields in the autopsy report, toxin report, cardiovascular report, or to infer one or more causes of death, the trained slide matching AI models may be able to output a cause of death or field(s) of a report. There may be multiple ways to build a system, as described below.

The system and methods described below may provide an alternative to requiring autopsies on dead humans or animals. The methods and techniques described herein may be implemented in a potentially less invasive and/or dignity-preserving manner in comparison to, for example, traditional necropsy or autopsy techniques. The system may implement information beyond the traditional methods of autopsy examination to be utilized, such as genetic information and toxin-related information. AI/ML tools applied to necropsy, in accordance with embodiments disclosed herein may democratize a non-universal process and provide a more complete understanding of causes of death.

As disclosed herein, various AI/ML tools applied to small samples of tissue extracted from a decedent and stored as WSIs can be used to extract information beyond that which a pathologist can evaluate. The AI/ML tools provided herein further offer a solution to some of the challenges posed by traditional autopsy methods.

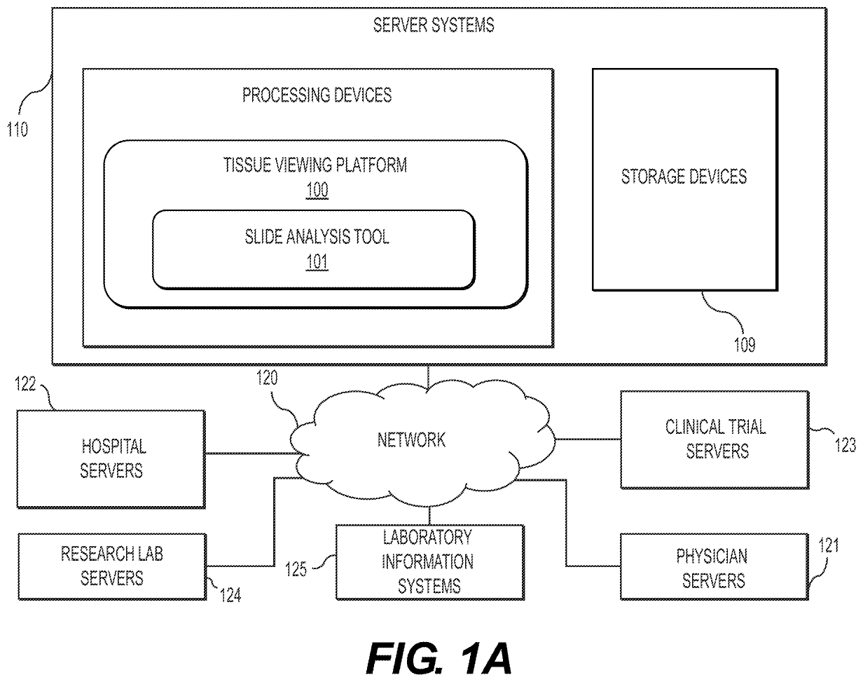

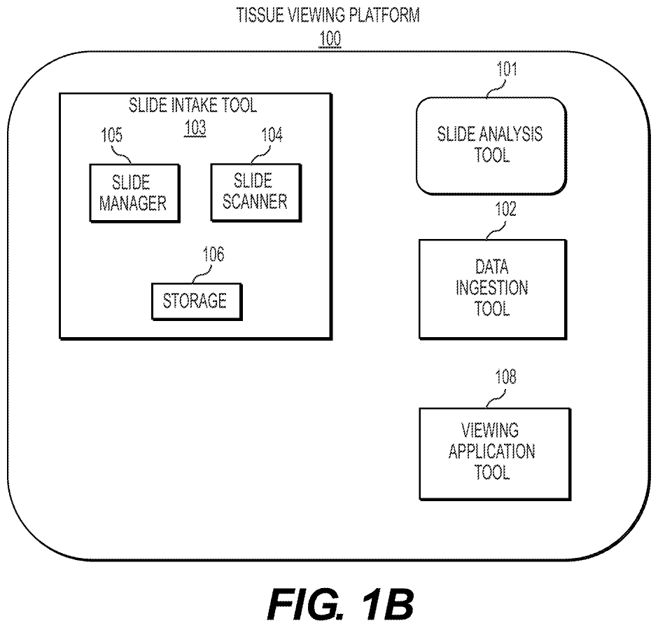

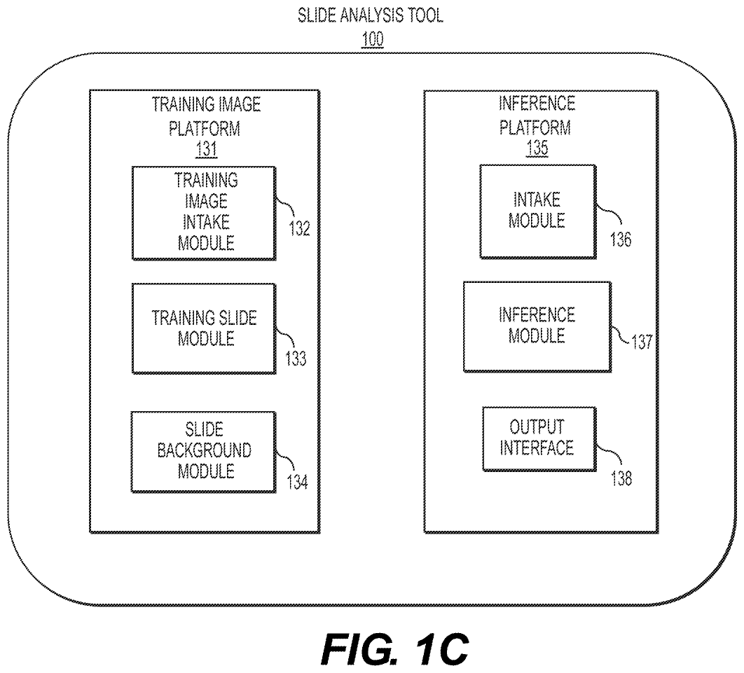

FIG. 1A illustrates a block diagram of a system and network for processing images to determine a cause of death, using machine learning, according to an exemplary embodiment of the present disclosure.

Specifically, FIG. 1A illustrates an electronic network 120 that may be connected to servers at hospitals, laboratories, and/or doctors' offices, etc. For example, physician servers 121, hospital servers 122, clinical trial servers 123, research lab servers 124, and/or laboratory information systems 125, etc., may each be connected to an electronic network 120, such as the Internet, through one or more computers, servers, and/or handheld mobile devices. According to an exemplary embodiment of the present disclosure, the electronic network 120 may also be connected to server systems 110, which may include processing devices that are configured to implement a tissue viewing platform 100, which includes a slide analysis tool 101 for determining specimen property or image property information pertaining to digital pathology image(s), and using machine learning to classify a specimen, according to an exemplary embodiment of the present disclosure.

The physician servers 121, hospital servers 122, clinical trial servers 123, research lab servers 124, and/or laboratory information systems 125 may create or otherwise obtain images of one or more patients' cytology specimen(s), histopathology specimen(s), slide(s) of the cytology specimen(s), digitized images of the slide(s) of the histopathology specimen(s), or any combination thereof. The physician servers 121, hospital servers 122, clinical trial servers 123, research lab servers 124, and/or laboratory information systems 125 may also obtain any combination of patient-specific information, such as age, medical history, cancer treatment history, family history, past biopsy or cytology information, etc. The physician servers 121, hospital servers 122, clinical trial servers 123, research lab servers 124, and/or laboratory information systems 125 may transmit digitized slide images and/or patient-specific information to server systems 110 over the electronic network 120. Server systems 110 may include one or more storage devices 109 for storing images and data received from at least one of the physician servers 121, hospital servers 122, clinical trial servers 123, research lab servers 124, and/or laboratory information systems 125. Server systems 110 may also include processing devices for processing images and data stored in the one or more storage devices 109. Server systems 110 may further include one or more machine learning tool(s) or capabilities. For example, the processing devices may include a machine learning tool for a tissue viewing platform 100, according to one embodiment. Alternatively or in addition, the present disclosure (or portions of the system and methods of the present disclosure) may be performed on a local processing device (e.g., a laptop).

The physician servers 121, hospital servers 122, clinical trial servers 123, research lab servers 124, and/or laboratory information systems 125 refer to systems used by pathologists for reviewing the images of the slides. In hospital settings, tissue type information may be stored in one of the laboratory information systems 125. However, the correct tissue classification information is not always paired with the image content. Additionally, even if a laboratory information system is used to access the specimen type for a digital pathology image, this label may be incorrect due to the face that many components of a laboratory information system may be manually input, leaving a large margin for error. According to an exemplary embodiment of the present disclosure, a specimen type may be identified without needing to access the laboratory information systems 125, or may be identified to possibly correct laboratory information systems 125. For example, a third party may be given anonym ized access to the image content without the corresponding specimen type label stored in the laboratory information system. Additionally, access to laboratory information system content may be limited due to its sensitive content.

FIG. 1B illustrates an exemplary block diagram of a tissue viewing platform 100 for determining specimen property of image property information pertaining to digital pathology image(s), using machine learning. For example, the tissue viewing platform 100 may include a slide analysis tool 101, a data ingestion tool 102, a slide intake tool 103, a slide scanner 104, a slide manager 105, a storage 106, and a viewing application tool 108.

The slide analysis tool 101, as described below, refers to a process and system for processing digital images associated with a tissue specimen, and using machine learning to analyze a slide, according to an exemplary embodiment.

The data ingestion tool 102 refers to a process and system for facilitating a transfer of the digital pathology images to the various tools, modules, components, and devices that are used for classifying and processing the digital pathology images, according to an exemplary embodiment.

The slide intake tool 103 refers to a process and system for scanning pathology images and converting them into a digital form, according to an exemplary embodiment. The slides may be scanned with slide scanner 104, and the slide manager 105 may process the images on the slides into digitized pathology images and store the digitized images in storage 106.

The viewing application tool 108 refers to a process and system for providing a user (e.g., a pathologist) with specimen property or image property information pertaining to digital pathology image(s), according to an exemplary embodiment. The information may be provided through various output interfaces (e.g., a screen, a monitor, a storage device, and/or a web browser, etc.).

The slide analysis tool 101, and each of its components, may transmit and/or receive digitized slide images and/or patient information to server systems 110, physician servers 121, hospital servers 122, clinical trial servers 123, research lab servers 124, and/or laboratory information systems 125 over an electronic network 120. Further, server systems 110 may include one or more storage devices 109 for storing images and data received from at least one of the slide analysis tool 101, the data ingestion tool 102, the slide intake tool 103, the slide scanner 104, the slide manager 105, and viewing application tool 108. Server systems 110 may also include processing devices for processing images and data stored in the storage devices. Server systems 110 may further include one or more machine learning tool(s) or capabilities, e.g., due to the processing devices. Alternatively or in addition, the present disclosure (or portions of the system and methods of the present disclosure) may be performed on a local processing device (e.g., a laptop).

Any of the above devices, tools and modules may be located on a device that may be connected to an electronic network 120, such as the Internet or a cloud service provider, through one or more computers, servers, and/or handheld mobile devices.

FIG. 1C illustrates an exemplary block diagram of a slide analysis tool 201, according to an exemplary embodiment of the present disclosure. The slide analysis tool may include a training image platform 131 and/or an inference platform 135.

The training image platform 131, according to one embodiment, may create or receive training images that are used to train a machine learning system to effectively analyze and classify digital pathology images. For example, the training images may be received from any one or any combination of the server systems 110, physician servers 121, hospital servers 122, clinical trial servers 123, research lab servers 124, and/or laboratory information systems 125. Images used for training may come from real sources (e.g., humans, animals, etc.) or may come from synthetic sources (e.g., graphics rendering engines, 3D models, etc.). Examples of digital pathology images may include (a) digitized slides stained with a variety of stains, such as (but not limited to) H&E, Hematoxylin alone, IHC, molecular pathology, etc.; and/or (b) digitized image samples from a 3D imaging device, such as micro-CT.

The training image intake module 132 may create or receive a dataset comprising one or more training images corresponding to either or both of images of a human and/or animal tissue and images that are graphically rendered. For example, the training images may be received from any one or any combination of the server systems 110, physician servers 121, and/or laboratory information systems 125. This dataset may be kept on a digital storage device. The training slide matching module 133 may intake training data related to a cause of death. For example, training slide module 133 training data may include receiving one or more images (e.g., WSIs) of a deceased human or animal. Further, the training data may include information such as age, ethnicity, and ancillary test results. The training data may also include biomarkers such as genomic/epigenomic/transcriptomic/proteomic/microbiome information can also be ingested, e.g., point mutations, fusion events, copy number variations, microsatellite instabilities (MSI), or tumor mutation burden (TMB). The training slide module 133 may intake full WSIs, or may intake one or more tiles of WSIs. The training slide module 133 may include the ability to break an inputted WSI into tiles to perform further analysis of individual tiles of a WSI. The training slide matching module 133 may utilize convolutional neural network (“CNN”), a Graph Neural Network (GNN), CoordConv, Capsule network, Random Forest Support Vector Machine, Transformer trained directly with the appropriate loss function in order to help provide training for the machine learning techniques described herein. Training slide module may also be used to train a machine learning module to determine a cause of death, predicts the fields on an autopsy report, cardiac arrhythmogenic genes, contributing factors of cardiovascular disease, liver toxins, and/or cause of miscarriage. The slide background module 134 may analyze images of tissues and determine a background within a digital pathology image. It is useful to identify a background within a digital pathology slide to ensure tissue segments are not overlooked.

According to one embodiment, the inference platform 135 may include an intake module 136, an inference module 137, and an output interface 138. The inference platform 135 may receive a plurality of electronic images/additional information and apply one or more machine learning model to the received plurality of electronic images/information to predicts the fields on an autopsy report, contributing causes of death, cardiac arrhythmogenic genes, contributing factors of cardiovascular disease, liver toxins, and/or cause of miscarriage. For example, the plurality of electronic images or additional information may be received from any one or any combination of the server systems 110, physician servers 121, hospital servers 122, clinical trial servers 123, research lab servers 124, and/or laboratory information systems 125. The intake module 136 may receive WSI's corresponding to one or more patients/individuals that may be deceased. The intake module 136 may further receive a gross description relating to one or more WSI. The gross description may contain information about the size, shape, and appearance of a specimen based on an examination of a WSI. The intake module 136 may further receive age, ethnicity, and ancillary test results and biomarkers such as genomic/epigenomic/transcriptomic/proteomic/microbiome information can also be ingested, e.g., point mutations, fusion events, copy number variations, microsatellite instabilities (MSI), or tumor mutation burden (TMB). The inference module 137 may apply one or more machine learning models to a group of WSI and any additional information in order to determine the fields on an autopsy report, contributing causes of death, cardiac arrhythmogenic genes, contributing factors of cardiovascular disease, liver toxins, and/or cause of miscarriage. The inference module 137 may further incorporate the spatial characteristics of the salient tissue into the prediction. For example, the inference module 137 may be capable of outputting one or more fields in an autopsy report or one or more contribution causes of death. The inference module 137 prediction may associate ordinal values, integer, or real numbers for each potential cause of death. The inference module 137 may further predict the likelihood of each potential cause of death based on the ordinal values, integers, or real numbers. Further, the inference module 137 may be capable of, when receive more than one WSI, determining and/or ranking which WSIs provide the most support for each cause of death. Further, the inference module 137 may be capable of indicating on WSIs the location of evidence that supports an autopsy report field, contributing causes of death, cardiac arrhythmogenic genes, contributing factors of cardiovascular disease, liver toxins, or cause of miscarriage.

The output interface 138 may be used to output information about the inputted images and additional information (e.g., to a screen, monitor, storage device, web browser, etc.). The output information may include information related to ranking causes of death. Further, output interface 138 may output WSI's that indicate locations/salient regions that include evidence related to outputs from inference module 137.

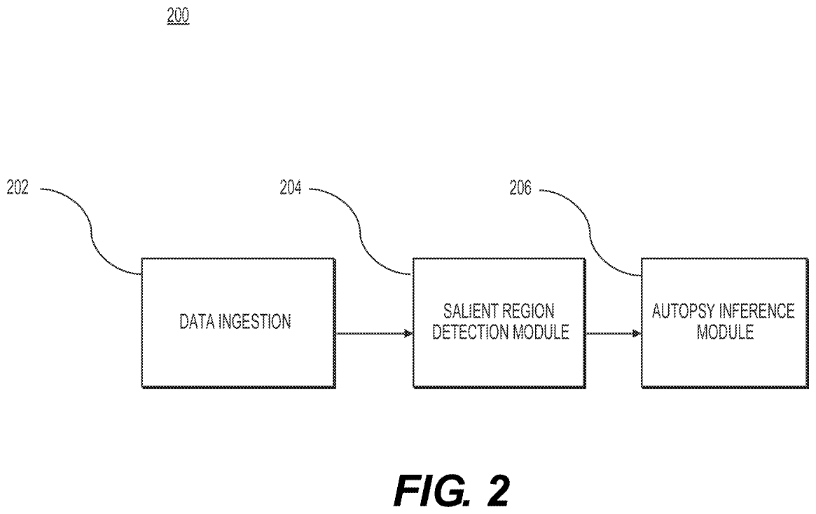

FIG. 2 illustrates a process for determining one or more contributing causes of death, one or more fields of autopsy report, cardiac arrhythmogenic genes, contributing factors of cardiovascular disease, liver toxins, and/or cause of miscarriage, according to techniques presented herein. In FIG. 2, the system may first include data ingestion 202. Data ingestion 202 may include receiving one or more digital medical images (e.g., whole slide image (WSI) of an autopsy pathology specimen, magnetic resonance imaging (MRI), computed tomography (CT), positron emission tomography (PET), mammogram, etc.) into a digital storage device (e.g., hard drive, network drive, cloud storage, RAM, etc.). Data ingestion 202 may further include deceased human/animal information, e.g., age, ethnicity, ancillary test results, and autopsy reports. Data ingestion 202 may also include biomarkers such as genomic/epigenomic/transcriptomic/proteomic/microbiome information, e.g., point mutations, fusion events, copy number variations, microsatellite instabilities (MSI), or tumor mutation burden (TMB). Each inputted image may be paired with the corresponding information during data ingestion 202.

Next, data ingested may be inserted into a salient region detection module 204, as described in greater detail below. A salient region detection module 204, as further described below, may be used to identify salient regions to be analyzed for each digital image. A salient region may be an image or one or more areas of an image that are considered relevant to a pathologist determining a diagnosis or treatment. A salient region may be dependent on what type of analysis is being performed on a slide. For example, when performing analysis of a whole slide image to determine which drug to utilize to target a cancer, the areas of a whole slide image that includes the cancerous region or the areas of the cancer that have the most immune cells (e.g., tumor infiltrating lymphoctyes) contained within the cancer may be considered the salient regions. A salient region may be specific to a particular application of the invention. Further, the salient region may be particular to specific organs or may depend on specific toxins. For example, for certain toxins, the exact morphology of the necrotic regions may be relevant to a diagnosis and thus be considered salient regions in an image. In another example, within kidney tissues, the salient region may be in and around the glomerulus. The detection of a salient region may be done manually by a user or may be done automatically using AI/ML. An entire image or specific image regions may be identified as salient. The entire disclosure of U.S. Non-Provisional application Ser. No. 17/313,617 filed May 6, 2021 is hereby incorporated herein by reference in its entirety.

Next, the digital whole slide images from the data ingestion 202, which may or not have had a salient region identified, may be provided to a pathology inference module 206. A pathology inference module, as further described below, may be used to infer one or more fields in one or more of an autopsy report, toxin report, cardiovascular report, or cause of death using machine learning and computer vision from the one or more digital image(s). The pathology inference module may incorporate spatial information from disparate regions in an image. A prediction may be output to an electronic storage device.

As discussed above, a salient region detection module 204 may be utilized prior to the system identifies a cause of death or field of an autopsy report. A salient region detection module 204 is further described herein. A continuous score of interest may be specific to certain structures within the digital image. The continuous score of interest may be a score assigned to one or more pixels to determine whether the one or more pixel should be considered within or part of a salient region. The system may determine a threshold value that one or more pixels or groups of pixels must surpass to be considered a salient region. In one embodiment, the score may range between 0 and 1, wherein a higher score may indicate that a region (e.g., set of pixels) is more likely to be a salient region and a low score indicates a region is not salient. It may be important to identify relevant regions so that they can be included, while excluding irrelevant ones during further analysis. For example, with MRI, PET, or CT data localizing a specific organ of interest may be useful/needed. Salient region identification may enable a downstream machine learning system to learn how to detect morphologies from less annotated data and to make more accurate predictions.

The salient region detection module 204 may output a salient region that was specified by a human annotator using an image segmentation mask, a bounding box, line segment, point annotation, freeform shape, or a polygon, or any combination of the aforementioned. Alternatively, this salient region detection module 204 may be created using machine learning to identify any appropriate locations that may be further examined.

Strong supervised methods and weakly supervised methods of machine learning may be used to create a salient region using the salient region detection module 204. Strongly supervised methods may identify, in a more precise manner, where the morphology of interest may be found. According to an implementation, a level of precision may be provided. For example, a probability or percentage may be provided for the likelihood of a given location having a morphology of interest may be outputted by salient region detection module 204. Weakly supervised methods might not provide precise locations, but rather output more general areas or specific medical digital images for further analysis.

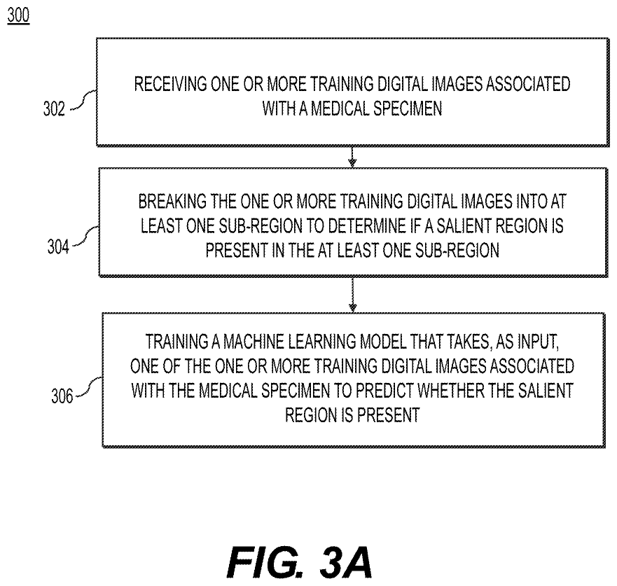

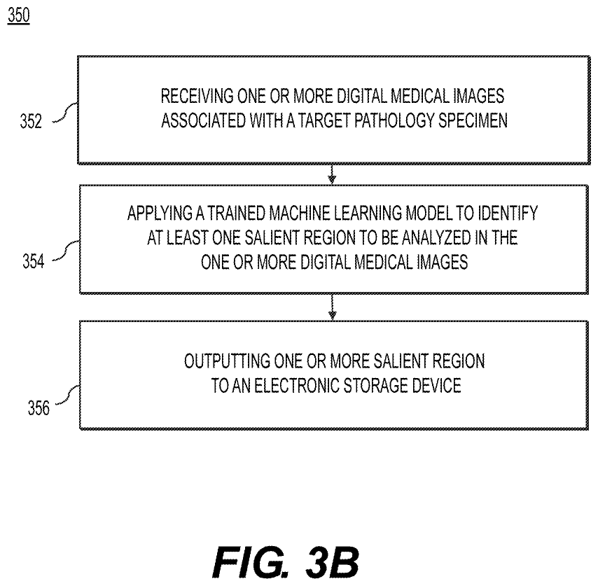

For strongly supervised training, the salient region detection module 204 may use an image and the location of the salient regions that could potentially express a biomarker as input. For two-dimensional (2D) images (e.g., whole slide images (WSI) in pathology), these locations could be specified with pixel-level labeling, bounding box-based labeling, polygon-based labeling, or using a corresponding image where the saliency has been identified (e.g., using IHC). For three-dimensional (3D) images (e.g., CT and MRI scans), the locations could be specified with voxel-level labeling, using a cuboid, etc., or use a parameterized representation allowing for subvoxel-level labeling, such as parameterized curves or surfaces, or deformed template. For weakly supervised training, the salient region detection module 204 may use the image or images and the presence/absence of the salient regions, but the exact location of the salient location might not be specified. Below, method 300 of FIG. 3A may describe one or more method of training salient region detection module 204 and method 350 of FIG. 3B may describe one or more method of using salient region detection module 204.

FIG. 3A is a flowchart illustrating an example method for training an algorithm for determining image salient region detection, according to techniques presented herein. The processes and techniques described in FIG. 3A may be used to train a machine learning model to identifier salient regions of medical digital images. The method 300 of FIG. 3A depicts steps that may be performed by, for example, training image platform 131 of slide analysis tool 101 as described above in FIG. 1C. Alternatively, the method may be performed by an external system.

Flowchart/method 300 depicts training steps to train a machine learning model as described in further detail in steps 302-306. The machine learning model may be used to identify salient regions of digital medical images as discussed further below.

At step 302, a system (e.g., the training image intake module 132 of slide analysis tool 101) may receive one or more digital images of a medical specimen (e.g., histology, CT, MRI, etc.) and further store the images within a digital storage device (e.g., hard drive, network drive, cloud storage, RAM, etc.). Further, the system may receive, for each inputted digital image, an indication of the presence or absence of the salient region (e.g., invasive cancer present, lymphovascular space invasion (LVSI), in situ cancer, etc.) within the image.

At step 304, each digital image may be broken into sub-regions that may then have their saliency determined. Saliency determination in various techniques discussed herein may be an optional step. Regions can be specified in a variety of methods, including creating tiles of the image, segmentations based on edge/contrast, segmentations via color differences, segmentations based on energy minimization, supervised determination by the machine learning model, EdgeBoxes, etc.

At step 306 a machine learning system may be trained that takes as input a digital image and predicts whether the salient region is present or not. Many methods may be used for the machine learning system to learn which regions are salient, including but not limited to (1) weak supervision, (2) bounding box or polygon-based supervisor, or (3) pixel-level or voxel-level labeling.

Weak supervision may include training a machine learning model (e.g., multi-layer perceptron (MLP), convolutional neural network (CNN), Transformers, graph neural network, support vector machine (SVM), random forest, etc.) using multiple instance learning (MIL) using weak labeling of the digital image or a collection of images. The label may correspond to the presence or absence of a salient region.

Bounding box or polygon-based supervision may include training a machine learning model (e.g., R-CNN, Faster R-CNN, Selective Search, etc.) using bounding boxes or polygons that specify the sub-regions of the digital image that are salient for the detection of the presence or absence of the biomarker.

Pixel-level or voxel-level labeling (e.g., a semantic or instance segmentation) may include training a machine learning model (e.g., Mask R-CNN, U-Net, Fully Convolutional Neural Network, Transformers, etc.) where individual pixels/voxels are identified as being salient for the detection of the continuous score(s) of interest. Labels may include in situ tumor, invasive tumor, tumor stroma, fat, etc. Pixel-level/voxel-level labeling can be from a human annotator or may be from registered images that indicate saliency.

FIG. 3B is a flowchart illustrating methods for image region detection, according to one or more exemplary embodiments herein. FIG. 3B may illustrate a method that utilizes the neural network that was trained in FIG. 3A. The exemplary method 350 (e.g., steps 352-356) of FIG. 3B depicts steps that may be performed by, for example, by inference platform 135 of slide analysis tool 101. These steps may be performed automatically or in response to a request from a user (e.g., physician, pathologist, etc.). Alternatively, the method described in flowchart 350 may be performed by any computer process system capable of receiving image inputs such as device 1100 and capable of including or importing the neural network described in FIG. 3A.

At step 352, a system (e.g., intake module 136 of slide analysis tool 101) may receive one or more digital medical images may be received of a medical specimen into a digital storage device (e.g., hard drive, network drive, cloud storage, RAM, etc.). Further, each digital image may be broken/divided into sub-regions using the techniques described in step 304. A saliency may be determined for one or more of the sub-regions (e.g., tissue for which the toxins should be identified) using the trained machine learning module from step 306.

At step 354, the trained machine learning system from FIG. 3A may be applied to the inputted images to predict which regions of the one or more images are salient and could potentially exhibit the continuous score(s) of interest (e.g., cancerous tissue). This application may include expanding the region to additional tissue, e.g., detecting an invasive tumor region, determining its spatial extent, and then extracting the stroma around the invasive tumor.

At step 356, if salient regions are found at step 354, the system may identify the salient region locations and flag them. If salient regions are present, detection of the region can be done using a variety of methods, including but not restricted to: running the machine learning model on image sub-regions to generate the prediction for each sub-region; or using machine learning visualization tools to create a detailed heatmap, etc., and then extracting the relevant regions.

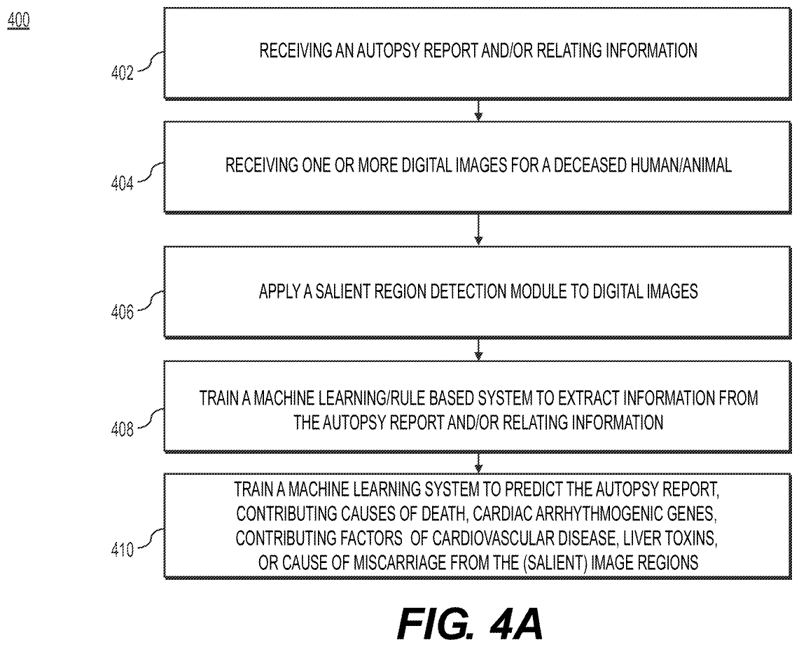

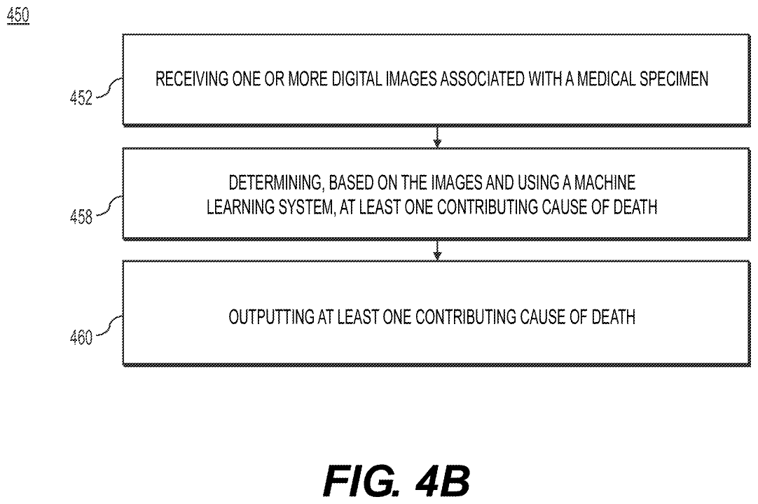

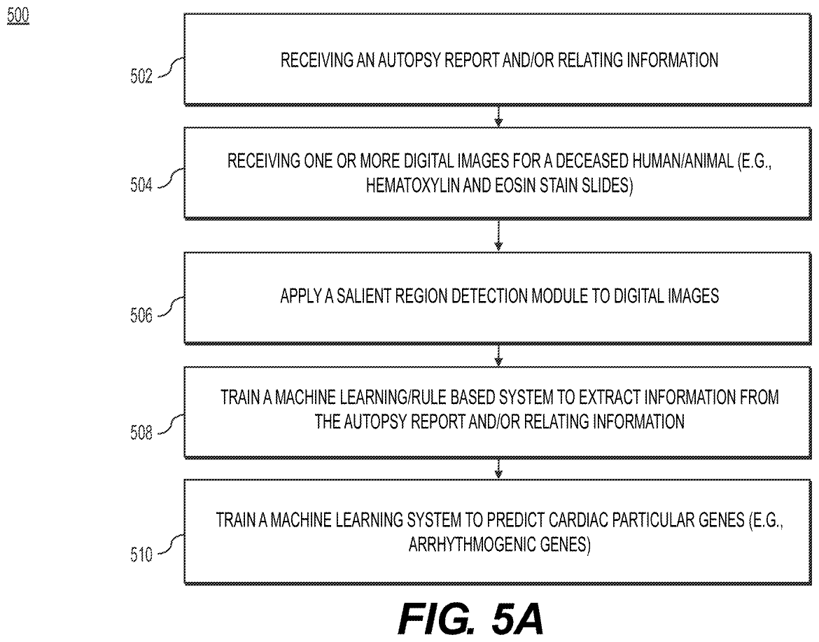

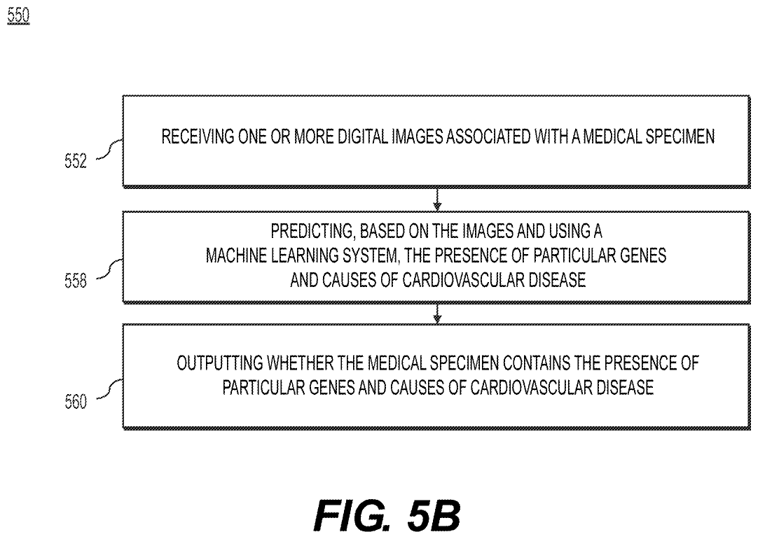

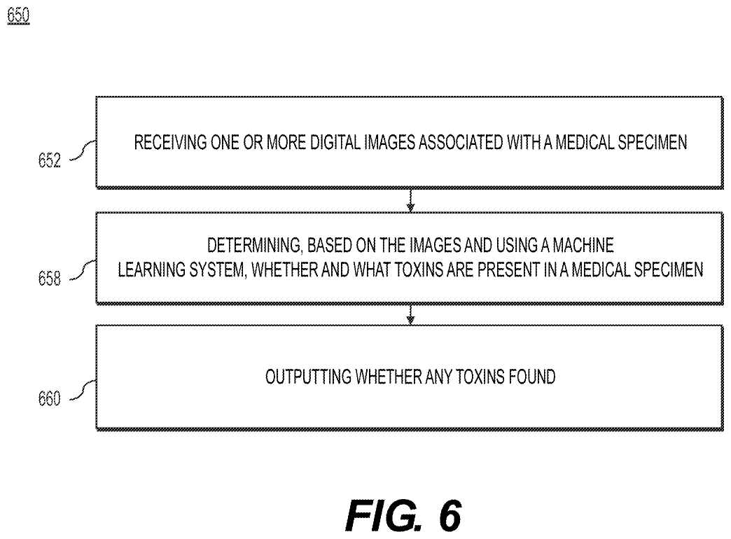

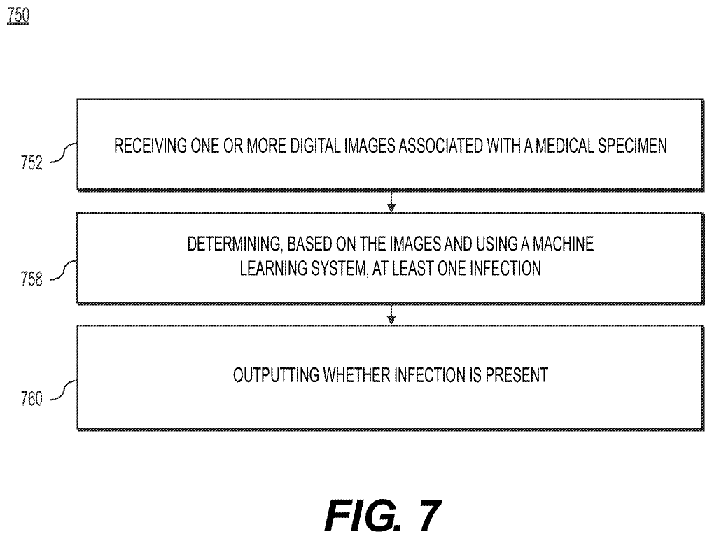

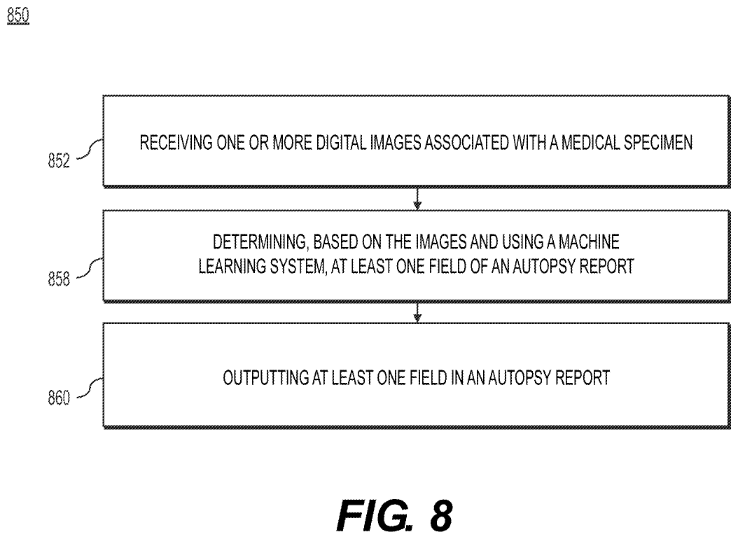

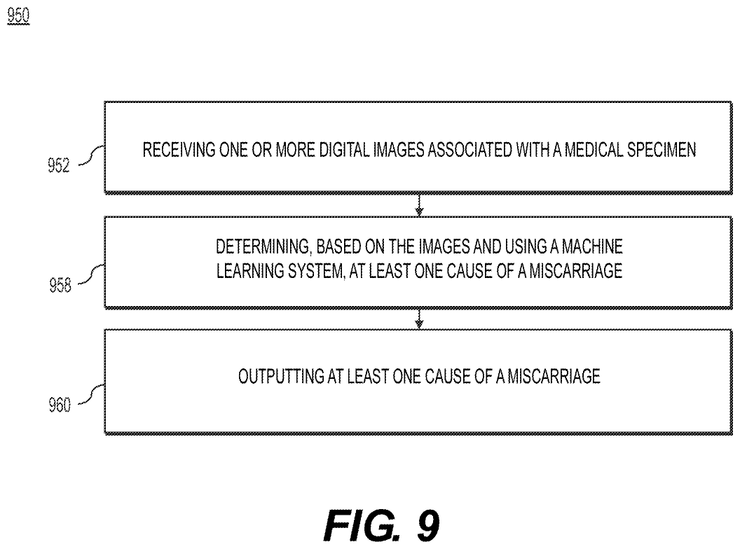

The outputted salient regions from step 356, may then be fed into the pathology inference module 206. The pathology inference module 206 may predict the fields on an autopsy report, contributing causes of death, cardiac arrhythmogenic genes, contributing factors of cardiovascular disease, liver toxins, cause of miscarriage, etc. and may incorporate spatial characteristics of the salient tissue into the prediction. Two techniques may be used alternatively or in combination to create a pathology inference module that uses spatial characteristics: 1) end-to-end, and/or 2) a two-stage prediction system. The end-to-end system may be trained directly from the input image whereas the two-stage system may first extract features from an image and then use machine learning methods that can incorporate the spatial organization of the features. The training of the pathology inference module 206 is described in greater detail below. Examples of training the pathology inference module 206 include method 400 of FIG. 4A and method 500 of FIG. 5A. Examples of using the pathology inference module 206 include method 450 of FIG. 5B, method 550 of FIG. 5B, method 650 of FIG. 6, method 750 of FIG. 7, method 850 of FIG. 8, and. method 950 of FIG. 9. The system may use the salient region module 204 as a part of training and using the pathology inference module 206.

FIG. 4A is a flowchart an example method for training an algorithm for determining one or more cause of death, according to techniques presented herein. The method 400 of FIG. 4A depicts steps that may be performed by, for example, training image platform 131 of slide analysis tool 101 as described above in FIG. 1C. Alternatively, the method 400 may be performed by an external system.

According to this embodiment, an AI/ML system may be built to predict and suggest potential contributing causes of death and supportive information to the physician conducting the autopsy. These contributing causes can also be ranked based on their likelihood prediction. This could apply to medical autopsies of human decedents, forensic autopsies of human decedents, and in the investigation of the death of any animal (i.e., rat, cat, horse, dog, monkey, etc.) in which the cause of death is unknown and of interest

Flowchart/method 400 depicts training steps to train a machine learning module as describe in further detail in steps 402-410.

At step 402, the system (e.g., the training image intake module 132 of slide analysis tool 101) may receive an autopsy report and/or related information. The autopsy report and related information may be either an electronically documented text paragraph, structured data, or numbers stored into a digital storage device (e.g., hard drive, network drive, cloud storage, RAM, etc.), and may be accessed via an anatomic pathology laboratory information system 125 (APLIS) (e.g., laboratory information system 125), digital evidence and forensics system or other digital systems. The system may further receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

At step 404, the system (e.g., the training image intake module 132 of slide analysis tool 101) may receive one or more digital images for a deceased human/animal into a digital storage device (e.g., hard drive, network drive, cloud storage, RAM, etc.). The digital image may be a whole slide image (“WSI”), which may refer to a digital image of a prepared microscopy slide. In particular, the system may receive hematoxylin and eosin (“H&E”) stained WSI. For training the machine learning system, each digital image may correspond to inputted autopsy reports and/or related information and/or gross descriptions. The system may further receive auxiliary non-image input variables, e.g., body temperature or external environmental temperature. The digital images received at step 404, for training, may each include corresponding data on the cause or causes of death for the particular individual. This data may be inputted at step 402 and may be information extracted from the autopsy report or gross description.

At step 406, the system (e.g., the training image platform 131 of slide analysis tool 101) may use the salient region detection module 204 to determine the saliency of each region within the image and exclude non-salient image regions from subsequent processing.

At step 408, the system may train a machine learning model or configure a rule-based system to extract the text of the autopsy report or other information if required. The machine learning system may use Natural Language Processing (NLP) systems such as encoder decoder systems, Seq2Seq and/or Recurrent Neural Networks to extract a structured form of the information. Given structured information such as a comma separated file (e.g., a gross description, synoptic pathology reports, toxicology report, radiology report, imaging report, autopsy report, or reports from other studies such as electrocardiogram reports), a rule-based extraction system may be used.

At step 410, the system (e.g., training module 133 of slide analysis tool 101) may be used to train a machine learning system to predict one or more causes of death from the (salient) image regions. Causes of death could be represented as a list of possible reasons for a death. The output may include a corresponding classification layer that outputs a vector of probabilities indicating which cause of is most likely. The classifier may be responsible for predicting the cause of death as well as assigning probabilities of each potential cause of death having occurred. Each point of the cause of death vector may relate to a specific cause of death. The system may train a machine learning system to use identify causes of death from the training data inputted at steps 402-404. The input of the autopsy report and/or gross report may be used to modulate processing. This could be done by transforming the optional information into an embedding (a vector of information that encodes the optional information) and then including it as input to the AI system, which could be done by concatenating it or something like using a context modulation layer (e.g., Feature-wise Linear Modulation (FiLM) layers in a neural network).

If deceased human/animal information (e.g., age) and or genomic/epigenomic/transcriptomic/proteomic/microbiome is also used as an input in addition to medical image data, then that may be provided/fed into the machine learning system as an additional input feature. The potential types of machine learning systems of step 410 that may be utilized by the overall system include but are not limited to a CNN/CoordConv/Capsule network/Random Forest/Support Vector Machine/Transformer trained directly with the appropriate loss function. The machine learning system of step 410 may be a different/separate machine learning system compared to the one utilized by salient region module 204.

Each cause of death may further include spatial information related the physical evidence for each cause. To incorporate the spatial information, the coordinates of each pixel/voxel may optionally be concatenated to each pixel/voxel. Alternatively, the coordinates may optionally be appended throughout processing (e.g., as done by the CoordConv algorithm). Alternatively, the machine learning algorithm could take spatial information into consideration passively by self-selecting regions in the input to process. In another embodiment, the system may utilize neural network introspection methods that indicate what caused the network to activate. This may include utilizing Class Activaiton Maps and GradCAM. These programs may act as post-hoc methods that highlight evidence that the neural network used during the initial prediction. Thus, the evidence utilized to create the cause of death predictions will be determined.

FIG. 4B is a flowchart illustrating exemplary methods for determine one or more causes of death, according to one or more exemplary embodiments herein. The exemplary method 450 (e.g., steps 452-456) of FIG. 4B depicts steps that may be performed by, for example, by inference platform 135 of slide analysis tool 101. These steps may be performed automatically or in response to a request from a user (e.g., physician, pathologist, etc.). Alternatively, the method described in flowchart 450 may be performed by any computer process system capable of receiving image inputs such as device 1100 and capable of including or importing the neural network described in FIG.4A.

At step 452, the system (e.g., the intake module 136 of slide analysis tool 101) may receive digital images of pathology specimens from a deceased human/animal may be received (e.g., histology, cytology, etc.) into a digital storage device (e.g., hard drive, network drive, cloud storage, RAM, etc.).

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system captures data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receive a only a gross description and not a gross description, the system may use machine learning to extract the text of the gross description. Given a structured gross description or autopsy report, a rule-based text extraction system may be used.

In one embodiment, the system might receive only digital images as an input. The system may be capable of utilizing the trained machine learning system from step 410 to determine the cause of death and relevant region detection.

At step 458, the system (e.g., the inference module 137 of slide analysis tool 101) may determine, based on the gross description and images, using a machine learning system, one or more contributing causes of death. The system may use the trained machine learning system from FIG. 4A to predict the cause of death vector values. Each cause of death may be a class in the vector. The vector may have K+1 total values. K may be equivalent to all potential causes of death and the +1 may represent the class for unknown cause of death. Each value may correspond to a potential cause of death. These values may be displayed in a viewing platform or stored digitally. Further, based on the value assigned to each potential cause of death, the system may be capable of determining a list of most likely causes of deaths and rank the potential outputs. In addition, the AI system may predict, on to a digital whole slide pathology image, where evidence for the autopsy report information is located. In addition, such information may be displayed as a written description (e.g., near the image).

In this embodiment, at step 458, the system may also be capable of keeping track and storing all evidence that is used by the system to determine a cause of death. The system may further be capable of sorting and providing a ranking to evidence that helped most for determining a cause of death. For example, if there are multiple images as input the system may identify the images that are most relevant to each prediction or cause of death, and may also highlight the regions within each image that support that prediction beyond a predetermined threshold. The system may keep track of all slides, or regions therein, that provide evidence that support a specific cause of death. Further, these slides may be ranked based on which slides provide the most amount of evidence for each prediction. The most lethal slide may be shown to a user (e.g., a pathologist).

For example, the system, after being fed input images and a gross description, may produce a list of all potential causes of death. This list may include a probability or score rating how likely the particular cause of death was. In addition, the system may output or display to a user the slide with most evidence for the highest cause of death. Lastly, this slide may be labeled by the system itself to depict the areas of the slide that provide evidence for the cause of death.

At step 460, the system (e.g., the output interface 138 of slide analysis tool 101) may output one or more causes of death. The output may be displayed as a ranking of most likely cause of death. For instance, the system may output a most likely cause of death, followed by a second most likely cause of death, followed by a third most likely cause of death, etc. Additionally, the system may correlate a cause of death to the corresponding organ and output what organ may be response for a death. For example, if the cause of death is determined to be cardiac arrest, the system may be capable of outputting that the organ responsible for the cause of death is the heart.

Outputting at least one cause of death may include saving the information to electronic storage such as digital evidence or forensic system, or displaying the results to a pathology. This information can be automatically digitally stored into a Laboratory Information Management System (LIMS), hospital information system (HIS) or digital evidence and forensics system. The system may be capable of notifying/alerting law enforcement or other personnel if the cause of death is of potential legal concern. For example, if the system determines that the cause of death was poison, the system may be capable of providing a notification to law enforcement. The notification may be through an automated phone call, text message, email, fax, or any other form of electronic communication.

Further, at step 460, the system may be capable of outputting which slides provided the most evidence for the ranked cause of deaths. The system may rank the inputted slides from step 452 as (1) most lethal slides, (2) second most lethal slide, (3) third most lethal slide, etc.

An example use of the system described in FIG. 4B is that the system may receive different tissues from a racehorse that died. The trained system may be capable of predicting which organ was most likely the cause of death. Further, the system could rank the potential causes based on likelihood and rank the inputted the slides based on evidence provided. For instance, the system could output: most likely cause of death is heart failure, second most likely cause of death is lung failure, and third most likely cause of death is gastric mucosa.

The system described in 4B may be utilized by law enforcement. The system may further help providing family closure, by providing previously undetermined causes of death. These techniques may provide and enhance dignity preservation. By solely examining small tissue samples, a human body may have significantly less lab processes performed on it, leaving the body in a more dignified state. Another useful aspect of this embodiment is that it may be used by drug companies to help determine cause of death during clinical studies of humans and animals. This may provide more accurate information to the drug companies and help studies become safer and more efficient.

FIG. 5A may provide an example of how to train an embodiment of the pathology inference module 206 that may be used for cardiac (heart) assessment for early deaths and is capable of predicting cardiac particular genes. FIG. 5B may provide an example of how to use an embodiment of the pathology inference module 206 that may be used for cardiac (heart) assessment for early deaths and is capable of predicting cardiac particular genes.

The embodiment described in FIGS. 5A and 5B may be useful when a younger individual (e.g., under 20, under 30, under 40, etc.), dies and there is a question of whether the heart was involved. The death of a young individual may trigger an inquiry regarding whether the individual's heart is involved. In such a scenario, a molecular testing panel comprised of cardiac arrhythmogenic genes may be performed to determine whether the sudden unexplained death (SUD) is related to genes associated with cardiac function. According to the NYMed Examiner, 40% of SUD cases had identifiable pathogenic/likely pathogenic variants or variants of uncertain significance that warrant further family and functional studies. As some molecular diseases are heritable, it could be argued that the medical examiner's office has a duty to identify these diseases and alert families about their presence.

According to an embodiment discussed in FIGS. 5A and 5B, the techniques disclosed herein may be used to predict a cohort of genes directly from H&E WSIs by training an AI/ML algorithm using data that expresses the genes in the cohort. Similar to other embodiments, this embodiment can also be used in other organisms such as racehorses. Cardiac muscle issues are a common cause of death in racehorses. Predicting the exact cause of death may improve horse-breeding programs

FIG. 5A is a flowchart illustrating how to train an algorithm to recognize a cardiovascular disease cause of death, according to techniques presented herein. The method 500 of FIG. 5A depicts steps that may be performed by, for example, training image platform 131 of slide analysis tool 101 as described above in FIG. 1C. Alternatively, the method 500 may be performed by an external system.

Flowchart/method 500 depicts training steps to train a machine learning module as describe in further detail in steps 502-510.

Step 502 may utilize techniques described in step 402. Further, the related information may include information about the individual such as blood pressure levels, statistics related to smoking, information on individual's weight, and individual's cholesterol levels. This information may be current and historic information for the individual.

At step 504, similar to step 404, the system (e.g., the training image intake module 132 of slide analysis tool 101) may receive one or more digital images for a deceased human/animal into a digital storage device (e.g., hard drive, network drive, cloud storage, RAM, etc.). In this embodiment, the system may further receive H&E stained slides for training. These training slides may correspond to deceased humans and/or animals who have expressed any combination of specific genes (e.g., arrhythmogenic genes) as a ground truth for training. The ground truth for training may refer to training slides of individuals with confirmed arrhythmogenic genes. These genes may have been separately confirmed by genetic testing. For training the machine learning system, each digital image may correspond to inputted autopsy reports and/or related information and /or gross descriptions. The system may further receive auxiliary non-image input variables, e.g., body temperature or external environmental temperature.

The list of cardiac Arrhythmia genes that the system may search for may include, but is not limited to: ABCC9 (12p12.1), AKAP9 (7q21.2), ANK2 (4q25-26), ASPH (8q12.3), CACNA1C (12p13.33), CACNA1D (3p21.1), CACNA2D1 (7q21.11), CACNB2 (10p12.33-12.31), CALM1 (14q32.11), CALM2 (2p21), CALM3 (19q13.32), CASQ2 (1p13.1), CAV3 (3p25.3), DPP6 (7q36.2), GJA5 (1q21.2), GPD1L (3p22.3), HCN4 (15q24.1), KCNA5 (12p13.32), KCND3 (1p13.2), KCNE1 (21q22.12), KCNE2 (21q22.11), KCNE3 (11q13.4), KCNE5 (Xg23), KCNH2 (7q36.1), KCNJ2 (17q24.3), KCNJ5 (11q24.3), KCNJ8 (12p12.1), KCNQ1 (11p15.5-15.4), LAMP2 (Xg24), LMNA (1q22), NPPA (1p36.22), PKP2 (12p11.21), PLN (6q22.31), PRKAG2 (7q36.1), RANGRF (17p13.1), SCN3B (11q24.1), SCN4B (11q23.3), SCN5A (3p22.2), SLMAP (3p14.3), SNTA1 (20q11.21), TNNT2 (1q32.1), TRDN (6q22.31), and/or TRPM4 (19q13.33).

Step 506 may utilize techniques described in step 406.

Step 508 may utilize techniques described in step 408.

At step 510, the system (e.g., training module 133 of slide analysis tool 101) may be used to train a machine learning system to predict the presence of any gene amplification from the training set in addition to the cause of death. Step 510 may utilize techniques described in step 410 in addition to training the machine learning to identify particular genes. The system may train a machine learning system to create a vector, where each potential value of the vector represents the chances of the individual having a particular cardiac Arrhythmia gene. The machine learning system may include a corresponding classification layer that outputs a vector of probabilities indicating particular percentage chances that particular genes exist in the individual. The classifier may be responsible for assigning probabilities of each potential Arrhythmia gene. Similar to step 410, the potential types of machine learning systems of step 510 that may be utilized by the overall system include but are not limited to a CNN/CoordConv/Capsule network/Random Forest/Support Vector Machine/Transformer trained directly with the appropriate loss function. The machine learning system of step 510 may be a different/separate machine learning system compared to the one utilized by salient region module 204. The system may be trained using images and their corresponding ground truths for particular genes that was provided at step 504.

FIG. 5B is a flowchart illustrating exemplary methods for determining one or more causes of death, according to one or more exemplary embodiments herein. The exemplary method 550 (e.g., steps 552-556) of FIG. 5B depicts steps that may be performed by, for example, by inference platform 135 of slide analysis tool 101. These steps may be performed automatically or in response to a request from a user (e.g., physician, pathologist, etc.). Alternatively, the method described in flowchart 550 may be performed by any computer process system capable of receiving image inputs such as device 1100 and capable of including or importing the neural network described in FIG. 5A.

At step 552 the system (e.g., the intake module 136 of slide analysis tool 101) may receive digital images (e.g., H&E whole slide images) of pathology specimens from a deceased human/animal may be received into a digital storage device (e.g., hard drive, network drive, cloud storage, RAM, etc.).

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system captures data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receives only a gross description and not a gross description, the system may use machine learning to extract the text of the gross description. Given a structured gross description or autopsy report, a rule-based text extraction system may be used. The system may further receive information about the individual such as blood pressure levels, statistics related to smoking, information on individual's weight, and individual's cholesterol levels. This information may be current and historic individual.

At step 558, the system (e.g., the inference module 137 of slide analysis tool 101) may determine, based on the input images of step 502, using a machine learning system, one or more potential cardiac arrhythmogenic genes separately or in addition to one or more contributing causes of death. The system may use the trained machine learning system from FIG. 5A to predict the cause of death and identify whether any cardiac arrhythmogenic genes are present. The output may be a vector that represents the chance that certain cardiac genes exist in an individual. This may be a separate vector than the cause of death vector discussed in step 458. With respect to the vector that outputs cardiac arrhythmogenic genes, the system may output that no genes are present unless the probability of any particular cardiag gene (e.g., a value of the vector) exceeds a threshold value. These values may be displayed in a viewing platform or stored digitally. The system may determine the percentage chance that a mutation in a cardiac arrhythomgenic gene contributed to a death based on the value assigned to each potential cardiac arrhythmogenic gene. The system may be capable of determining a list of most likely causes of deaths and rank the potential outputs. In addition, the AI system may predict, on to a digital whole slide pathology image, where evidence for cardiac arrhythmogenic genes and or other causes of death are located. Additionally, similar to the cause of death machine learning system, the system may keep track of and rank the slides based on what slides provide the most evidence that a certain gene is present. For each cardiac arrhythomgenic gene found (e.g., the value is above a certain threshold value), there may be a corresponding lists of slides that provide evidence for the presence of the gene. Alternatively, or in addition, such information may be displayed as a written description (e.g., near the image).

At step 560, the system may output a multi label prediction for the root cause of one or more cardiovascular disease. The highest root cause of cardiovascular events such as high blood pressure, high blood cholesterol, diabetes, obesity, smoking, or predict a mutated gene from a set of cardiac arrhythmogenic genes associated with SUD may then be displayed. For example, the contributing factor output of the system may be: 50% high blood pressure, 30% smoking, 10% obesity, 9% high blood cholesterol, <1% mutation in cardiac arrhythmogenic gene(s). Separately, the system may output a list of any potential arrhythmogenic gene(s).

FIG. 6 illustrates exemplary usage of modules such as the pathology inference module 206 for liver toxin analysis.

The metabolism of drugs and toxins occurs primarily in the liver, a large organ easily accessible by percutaneous biopsy under imaging guidance. Traditionally, at autopsy, toxicology is performed on blood samples taken from the decedent to determine if toxin exposure contributed to the manner and cause of death, only on cases where such a manner/cause is suspected. This is because it is expensive and time consuming, but with the techniques disclosed herein, this analysis could be done routinely.

This embodiment may use the techniques disclosed herein to assess H&E WSI from a liver biopsy sample to evaluate for the presence of toxins, either intentionally or accidentally ingested (e.g., heavy metals, rodenticides), or drugs. This technique may circumvent the need for opening of the abdominal cavity to sample the liver since it can be accessed by needle biopsy percutaneously. It may also allow universal toxin screening on all biopsies rather than an expensive, costly, and technically challenging blood test not performed at all institutions. Based on the results of the AI/ML screen, a more specific toxin assay may be performed. In cases of animal autopsies by drug companies performing clinical trials, this tool may determine whether the drug administered to the animal ultimately contributed to the animal's death.

The machine learning module utilized in FIG. 6 may be trained using techniques described in relation to FIG. 4A. Specifically, the machine learning module may be trained in this embodiment to determine, based on H&E WSI from a liver biopsy, whether the presence of toxins is included. The system may train a machine learning system to output a vector, wherein each point of the vector represents a potential toxin. The training may include the machine learning system receiving inputs of various training slides that may include H&E WSI from liver biopsies with corresponding identifiers as to which toxins are present in the training slides. Then, any of the potential machine learning systems described in FIG. 4A, or elsewhere herein, may be trained to output the whether toxins are present in slides.

Further, the salient region module applied at step 204 may be used prior to detecting toxins in order to determine the regions of the WSI from a liver biopsy related to presence of toxins. For example, depending on the type of toxin, different hepatic histological compartments may be a part of a salient region in this embodiment. In one example, when examining the effects of acetaminophen, the salient region may be within the hepatic lobule (e.g., to look for hepatic centrilobular necrosis).

FIG. 6 is a flowchart illustrating methods to, for example, determine any liver toxins and one or more causes of death, according to one or more exemplary embodiments herein. In one embodiment, the system may solely be used to determine the presence of liver toxins and not a cause of death. The exemplary method 650 (e.g., steps 652-660) of FIG. 6 depicts steps that may be performed by, for example, by inference platform 135 of slide analysis tool 101. These steps may be performed automatically or in response to a request from a user (e.g., physician, pathologist, etc.). Alternatively, the method described in flowchart 650 may be performed by any computer process system capable of receiving image inputs such as device 1100 and capable of including or importing the neural network described in FIG. 6.

At step 652 the system (e.g., the intake module 136 of slide analysis tool 101) may receive digital images (e.g., H&E whole slide images) of pathology specimens from a deceased (and/or living) human/animal may be received into a digital storage device (e.g., hard drive, network drive, cloud storage, RAM, etc.).

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system captures data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receive a only a gross description and not a gross description, the system may use machine learning to extract the text of the gross description. Given a structured gross description or autopsy report, a rule-based text extraction system may be used. At step 658, the system (e.g., the inference module 137 of slide analysis tool 101) may determine, based on the gross description and H&E whole slide images, using a machine learning system, whether any toxin (regardless of whether intentional or accidentally ingested) is/was present in the individual. The system may use the trained machine learning system from FIG. 4A to predict the presence of one or more liver toxins. Each value of the vector determined by the machine learning system may correspond to represent a specific toxin that may found within a human or animals. These values may be displayed in a viewing platform or stored digitally. Further, based on the value assigned to potential liver toxin, the system may be capable of determining whether any liver toxins were present, which toxins, and may determine whether the toxin contributed to the cause of death. In addition, the AI system may predict, on to a digital whole slide pathology image, where evidence for the autopsy report information is located. Alternatively, or in addition, such information may be displayed as a written description (e.g., near the image).

In this embodiment, at step 658, the system may also be capable of keeping track of and storing all evidence that is used by the system to determine a cause of death. The system may further be capable of sorting and providing a ranking to evidence that helped most for determining a cause of death. For example, if there are multiple images as input it can identify the images that are most relevant to each prediction, and may also highlight the regions within each image that support that prediction beyond a predetermined threshold. The most lethal slide may be shown to a user (e.g., a pathologist).

At step 660, the system (e.g., the output interface 138 of slide analysis tool 101) may output whether one or more toxins were present from a WSI (e.g., an H&E WSI from a liver biopsy). The output may be displayed as a list of any toxins found. Further, the system may rank the list based on highest amount of toxin present and/or by more dangerous toxin present. Outputting the toxins may further include saving the information to electronic storage such as digital evidence or forensic system, or displaying the results to a pathology. This information can be automatically digitally stored into a Laboratory Information Management System (LIMS), hospital information system (HIS) or digital evidence and forensics system.

FIG. 7 may provide an example of how to use an embodiment of the pathology inference module 206 that may be used for infection detection analysis.

Occult infection at death can be ascertained during an autopsy by examining blood and tissue cultures that assess for the growth of fungal or bacterial organisms, or by direct sampling and visualization of tissue containing these organisms. The embodiments disclosed herein and further described in method 750 may be configured to assess H&E WSI for the presence of fungi, bacteria, or mycobacteria. This may obviate the reliance on examining time-consuming and imprecise blood and tissue cultures for establishing a cause of infection.

The machine learning module utilized in FIG. 7 may be trained based on techniques described in relation to FIG. 4A. Specifically, the machine learning module may be trained in this embodiment to determine, based on H&E WSI, whether the presence of an infection is included. H&E slides with labeled infections may be fed to the machine learning model of FIG. 4A to train the system to output one or more infections found on a WSI. Looking for infection may include searching for the presence of fungi, bacteria, or mycobacteria.

FIG. 7 is a flowchart illustrating an exemplary method to determine one or more cause of death, according to one or more exemplary embodiments herein. The exemplary method 750 (e.g., steps 752-760) of FIG. 7 depicts steps that may be performed by, for example, by inference platform 135 of slide analysis tool 101. These steps may be performed automatically or in response to a request from a user (e.g., physician, pathologist, etc.). Alternatively, the method described in flowchart 750 may be performed by any computer process system capable of receiving image inputs such as device 1100 and capable of including or importing the neural network described in FIG. 7.

At step 752 the system (e.g., the intake module 136 of slide analysis tool 101) may receive digital images (e.g., H&E whole slide images) of pathology specimens from a deceased human/animal may be received into a digital storage device (e.g., hard drive, network drive, cloud storage, RAM, etc.).

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system captures data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receives only a gross description and not a gross description, the system may use machine learning to extract the text of the gross description. Given a structured gross description or autopsy report, a rule-based text extraction system may be used. At step 758, the system (e.g., the inference module 137 of slide analysis tool 101) may determine, based on the gross description and H&E whole slide images, using a machine learning system, whether infection is present. The system may use the trained machine learning system from FIG. 4A to predict whether an infection is present on an inserted WSI. The machine learning system may be capable of outputting a vector where each value of the vector corresponds to a particular potential infection. These values may be displayed in a viewing platform or stored digitally. Further, based on the value assigned to potential infectious diseases, the system may be capable of determining a list of whether any infectious diseases were present and determining whether the infection contributed to the cause of death. In addition, the AI system may predict, on to a digital whole slide pathology image, where evidence for the infectious disease information is located. Alternatively, or in addition, such information may be displayed as a written description (e.g., near the image).

In this embodiment, at step 758, the system may also be capable of keeping track and storing all evidence that is used by the system to determine whether an infectious disease is present. The system may further be capable of sorting and providing a ranking of evidence that helped most for determining whether an infectious disease was present. For example, if there are multiple images as input it can identify the images that are most relevant to each prediction, and may also highlight the regions within each image that support that prediction beyond a predetermined threshold. The slide that provides the most evidence may be shown/output to a user (e.g., a pathologist) at step 760.

At step 760, the system (e.g., the output interface 138 of slide analysis tool 101) may output whether one or more toxins were present from a WSI (e.g., an H&E WSI). The output may be displayed as a list of any infectious found. Further, the system may rank the list based on highest amount of infectious disease present and/or by more dangerous toxin present. Outputting the infectious disease may further include saving the information to electronic storage such as digital evidence or forensic system, or displaying the results to a pathology. This information can be automatically digitally stored into a Laboratory Information Management System (LIMS), hospital information system (HIS) or digital evidence and forensics system

FIG. 8 may provide an example of how to use an embodiment of the pathology inference module 206 that may be used to infer one or more fields in an autopsy report.

In many deaths, the precise time that death occurs may be unknown. In these cases, forensic analysis can be used to estimate the time of death, but this may be an imprecise process that involves integrating gross findings with environmental factors related to the location of the corpse. At the same time, time of death is a key piece of information in establishing the manner of death and in cases of law enforcement, the party responsible for death.

Embodiments of the disclosed subject matter may be used to address this problem. According to an embodiment described in method 850, the system may be trained using medical images where the time of death is known, allowing it to directly infer the time of death after training from new medical images not used for training. In addition, the system can receive additional non-image input variables such as the external environmental temperature and temperature of the corpse at the time of discovery. The external temperature recording can help the system to generalize to extreme cold or hot conditions. Furthermore, the system can predict other fields from the autopsy report such as the manner of death: accident, natural etc.

The machine learning module utilized in FIG. 8 may be trained based on FIG. 4A. Specifically, the machine learning module may be trained in this embodiment to determine a time of death, based on environmental temperature, corpse temperature at time of discovery, and WSIs.

FIG. 8 is a flowchart illustrating exemplary methods for determining the time of death and/or predicting additional fields from the autopsy report, according to one or more exemplary embodiments herein. The exemplary method 850 (e.g., steps 852-860) of FIG. 8 depicts steps that may be performed by, for example, by inference platform 135 of slide analysis tool 101. These steps may be performed automatically or in response to a request from a user (e.g., physician, pathologist, etc.). Alternatively, the method described in flowchart 850 may be performed by any computer process system capable of receiving image inputs such as device 1100 and capable of including or importing the neural network described in FIG. 8.

At step 852 the system (e.g., the intake module 136 of slide analysis tool 101) may receive digital images (e.g., H&E whole slide images) of pathology specimens from a deceased human/animal may be received into a digital storage device (e.g., hard drive, network drive, cloud storage, RAM, etc.).

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system may capture data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receive a only a gross description and not a gross description, the system may use machine learning to extract the text of the gross description. Given a structured gross description or autopsy report, a rule-based text extraction system may be used. Further, the system may receive external environmental temperature information and/or temperature of the corpse at the time of discovery.

At step 858, the system (e.g., the inference module 137 of slide analysis tool 101) may determine, based on the temperature information, gross description, and H&E whole slide images, using a machine learning system, what time a death occurred. The system may use the trained machine learning system from the description associated with FIG. 4A to predict a time of death value. The values may correspond to a time of death. These values may be displayed in a viewing platform or stored digitally. Further, based on the value assigned to the potential time of death, the system may be capable of determining a likelihood that a death occurred within a specific range of time. For example, the system may determine that there was an 80% chance that a death occurred between 4pm and 5pm on a particular date. In addition, the AI system may predict, on to a digital whole slide pathology image, where evidence for the liver toxins is located. Alternatively, or in addition, such information may be displayed as a written description (e.g., near the image).

In this embodiment, at step 658, the system may also be capable of keeping track and storing all evidence that is used by the system to determine when the time of death occurred. The system may further be capable of sorting and providing a ranking to evidence that helped most for determining when the time of death occurred. For example, if there are multiple images as input it can identify the images that are most relevant to each prediction, and may also highlight the regions within each image that support that prediction beyond a predetermined threshold. The slide or information (e.g., the temperatures) that provides the most information may be shown to a user at step 600.

At step 660, the system (e.g., the output interface 138 of slide analysis tool 101) may output a time or time range, within a predetermined confidence threshold, that death occurred. The system may further output a certainty level associated with the time. Outputting the time may further include saving the information to electronic storage such as digital evidence or forensic system, or displaying the results to a pathology. Further, the system may be capable of outputting the time of death specifically as a field of an autopsy report. This information can be automatically digitally stored in a Laboratory Information Management System (LIMS), hospital information system (HIS) and/or digital evidence and forensics system.

FIG. 9 provides an example of how an embodiment of the pathology inference module 206 may be used to predict a cause of a miscarriage or stillbirth.

Techniques discussed herein may be used to predict the cause of miscarriage by training the system using image data and/or metadata. The metadata may include genetic analysis performed on tissue of after a miscarriage or stillbirth. The process may include training the system using a collection of images from human/animals who have expressed any combination of these miscarriage causes as ground truth. Training may be performed using images and/or images of samples from the mother and/or images of the miscarried fetus, retained products of conception (RPOC), placenta, pregnancy sac, etc. Potential miscarriage causes include, but are not limited to, genetic abnormalities, drug or alcohol usage, abnormalities of the uterus, medical condition such as thyroid disease and diabetes, infections, hormonal problems, response of immune system, and cervical insufficiency. Once the system has been trained to predict the presence of any miscarriage cause from the training set, the model may be used to predict the presence of any of the miscarriage causes in other images. The system may be able to predict and potentially also rank the presence of genetic anomalies, infections, tubal pregnancies, amniocentesis and placenta dysfunction. Furthermore, the system may also be trained to predict environmental factors that caused the miscarriage such as drugs, alcohol, or tobacco.

The machine learning module utilized in FIG. 9 may be trained based on techniques discussed in relation to FIG. 4A. Specifically, the machine learning module may be trained in this embodiment to determine, based on metadata and digital images, whether miscarriage causes are present. The system may train the machine learning system to output a vector wherein each place of the vector represents a potential cause of a miscarriage.

FIG. 9 is a flowchart illustrating exemplary methods for determining one or more causes of miscarriage), according to one or more exemplary embodiments herein. The exemplary method 950 (e.g., steps 952-960) of FIG. 9 depicts steps that may be performed by, for example, by inference platform 135 of slide analysis tool 101. These steps may be performed automatically or in response to a request from a user (e.g., physician, pathologist, etc.). Alternatively, the method described in flowchart 950 may be performed by any computer process system capable of receiving image inputs such as device 1100 and capable of including or importing the neural network described in FIG. 9.

At step 952 the system (e.g., the intake module 136 of slide analysis tool 101) may receive digital images (e.g., H&E whole slide images) of pathology specimens from an individual that has had a miscarriage, stillbirth, trauma while pregnant. These digital images may be received into a digital storage device (e.g., hard drive, network drive, cloud storage, RAM, etc.). Additionally, the system may receive ultrasonographic findings from the pregnant mother that where taken while the live fetus was in utero.

In one embodiment, the system (e.g., the intake module 136 of slide analysis tool 101) may receive a gross description. The system may further receive deceased human/animal information (e.g., age, ethnicity, ancillary test results, etc.) that may be ingested to stratify and split the system for machine learning. The system may additionally ingest biomarkers such as genomic/epigenomic/transcriptomic/proteomic/microbiome information, e.g., point mutations, fusion events, copy number variations, microsatellite instabilities (MSI), or tumor mutation burden (TMB).

In one embodiment, the system may determine the metadata from one or more gross descriptions. The system may use a machine learning system or configured rule-based system to extract the text of the gross description of the tissue. This system captures data about the size, texture, color, shape, lesions, landmarks, and/or distances. The machine learning system may use trained Natural Language Processing (NLP) models such as encoder decoder systems, Seq2Seq and/or Recurrent Neural Networks to extract a structured form of the gross description. If the system receive a only a gross description and not a gross description, the system may use machine learning to extract the text of the gross description. Given a structured gross description or autopsy report, a rule-based text extraction system may be used. At step 955, the system may receive additional information related to an individual's drug or alcohol use.

At step 958, the system (e.g., the inference module 137 of slide analysis tool 101) may determine, based on the metadata and digital images, using a machine learning system, one or more causes of a miscarriage. The system may use the trained machine learning system described in relation to FIG. 4A to predict values of a vector that represent causes of a miscariage. These values may be displayed in a viewing platform or stored digitally. Further, based on the value assigned to causes of miscarriage, the system may be capable of determining a list of potential causes of miscarriage. Further, the potential causes of miscarriage may be ranked in order of likelihood of causing the miscarriage. In addition, the AI system may predict, on to a digital whole slide pathology image, where evidence for the one or miscarriage causes is located. Alternatively, or in addition, such information may be displayed as a written description (e.g., near the image).

In this embodiment, at step 958, the system may also be capable of keeping track and storing all evidence that is used by the system to determine a cause of miscarriage. The system may further be capable of sorting and providing a ranking to evidence that helped most for determining a cause of miscarriage. For example, if there are multiple images as input, the system may identify the images that are most relevant to each prediction, and may also highlight the regions within each image that support that prediction. One or more slides that provide the most evidence may be shown to a user (e.g., a pathologist).

At step 960, the system (e.g., the output interface 138 of slide analysis tool 101) may output the one or more cause of miscarriage. The output may be displayed as a list of potential causes ranked by likelihood. Outputting the causes of miscarriage may further include saving the information to electronic storage such as digital evidence or forensic system, or displaying the results to a pathology. This information can be automatically digitally stored into a Laboratory Information Management System (LIMS), hospital information system (HIS) and/or digital evidence and forensics system.

An embodiment of the disclosed subject matter may be used to optimize dog, racehorse, and other breeding programs. By detecting the cause of death of various organisms, breeders are able to catch and mitigate issues in their breeding programs early. According to this embodiment, early inefficiencies, or heart issues can be caught before a breeder continues with a specific line. The input to training such a system could be a binary value 0 or 1 indicating whether to continue breeding the offspring of the diseased organism. The value 1 would suggest continuation. The prediction could be electronically transmitted to the breeder.

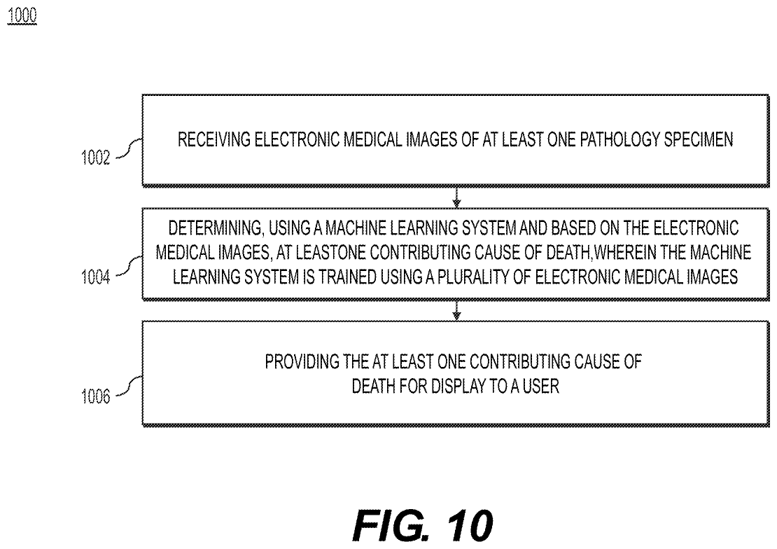

FIG. 10 is a flowchart illustrating exemplary methods for providing a contributing cause of death, according to one or more exemplary embodiments herein. Flowchart 1000 may depicts steps to utilize a trained machine learning module as describe in further detail in steps 1002-1010.

At step 1002, the system (e.g., the image intake module 136) may receive images of at least one pathology specimen, the pathology specimen being associated with an individual/patient.

At step 1004, the system (e.g., the inference module 137) may determine, using a machine learning system and based on the electronic medical images, at least one contributing cause of death, wherein the machine learning system is trained using a plurality of electronic medical images.

At 1006, the system (e.g., the output interface 138) may provide at least one contributing cause of death for display to a user.

In one embodiment, the system may further receive an autopsy report and information relating to an age, ethnicity, ancillary test results, and/or an autopsy report of the patient. The system may further detect one or more salient regions of each of the plurality of electronic medical images. The system may only have the machine learning system analyze the salient regions of the plurality of electronic medical images. Further, the machine learning systems may determine a numerical value score for each contributing cause of death. The system may also include marking the plurality of medical images to device where evidence for the contributing cause of death is located. When the system detects more than one contributing cause of death, the system may rank each contributing cause of death from most to least likely. The system may determine and rank which of the plurality of medical images provides the most evidence for the contributing cause of death.

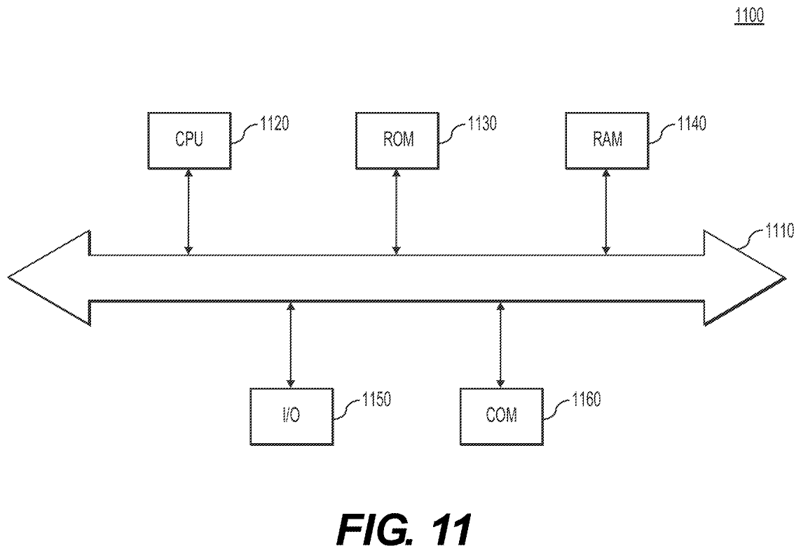

As shown in FIG. 11, device 1100 may include a central processing unit (CPU) 1120. CPU 1120 may be any type of processor device including, for example, any type of special purpose or a general-purpose microprocessor device. As will be appreciated by persons skilled in the relevant art, CPU 1120 also may be a single processor in a multi-core/multiprocessor system, such system operating alone, or in a cluster of computing devices operating in a cluster or server farm. CPU 1120 may be connected to a data communication infrastructure 1110, for example a bus, message queue, network, or multi-core message-passing scheme.

Device 1100 may also include a main memory 1140, for example, random access memory (RAM), and also may include a secondary memory 1130. Secondary memory 1130, for example a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1130 may include similar means for allowing computer programs or other instructions to be loaded into device 1100. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 1100.

Device 1100 also may include a communications interface (“COM”) 1160. Communications interface 1160 allows software and data to be transferred between device 1100 and external devices. Communications interface 1160 may include a modem, a network interface (such as an Ethernet card), a communications port, a PCMCIA slot and card, or the like. Software and data transferred via communications interface 1160 may be in the form of signals, which may be electronic, electromagnetic, optical or other signals capable of being received by communications interface 1160. These signals may be provided to communications interface 1160 via a communications path of device 1100, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 1100 may also include input and output ports 1150 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically can be grouped together to perform a function or group of related functions. Like reference numerals are generally intended to refer to the same or similar components. Components and modules may be implemented in software, hardware or a combination of software and hardware.

The tools, modules, and functions described above may be performed by one or more processors. “Storage” type media may include any or all of the tangible memory of the computers, processors, or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments of the invention will be apparent to those skilled in the art from consideration of the specification and practice of the invention disclosed herein. It is intended that the specification and examples to be considered as exemplary only.

What is claimed is:

1. A computer-implemented method for processing electronic medical images, comprising:
receiving a plurality of electronic medical images of at least one pathology specimen, the pathology specimen being associated with a patient;
determining, using a machine learning system and based on the electronic medical images, at least one contributing cause of death, wherein the machine learning system is trained using a plurality of electronic medical images; and
providing the at least one contributing cause of death for display to a user.

2. The method of claim 1, further including receiving an autopsy report and information relating to an age, ethnicity, ancillary test results, and/or an autopsy report of the patient.

3. The method of claim 1, further including detecting one or more salient region of each of the plurality of electronic medical images.

4. The method of claim 3, wherein the machine learning system only analyzes the salient regions of the plurality of electronic medical images.

5. The method of claim 1, wherein the machine learning system determines a numerical value score for each contributing cause of death.

6. The method of claim 1, further including marking the plurality of medical images to depict where evidence for the contributing cause of death is located.

7. The method of claim 1, wherein when more than one contributing cause of death is determined, ranking each contributing cause of death from most to least likely.

8. The method of claim 1, including determining and ranking which of the plurality of medical images provides the most evidence for the contributing cause of death.

9. The method of claim 1, including predicting, through the machine learning system, an organ that likely caused the death of the patient.

10. The method of claim 1, further comprising:
receiving a gross description, the gross description comprising data about the patient;
determining report metadata based on the gross description; and
wherein, the machine learning model uses the metadata and gross description, in addition to the received plurality of electronic medical images to predict a cause of death.

11. The method of claim 1, wherein the machine learning system outputs a vector and each place of the vector represents a potential cause of death, wherein each place of the vector represents a percent chance of a particular cause of death.

12. A system for processing electronic medical images, the system comprising:
at least one memory storing instructions; and
at least one processor configured to execute the instructions to perform operations comprising:
receiving a plurality of electronic medical images of at least one pathology specimen, the pathology specimen being associated with a patient;
determining, using a machine learning system and based on the electronic medical images, at least on contributing cause of death, wherein the machine learning system is trained using a plurality of electronic medical images; and
providing the at least one contributing cause of death for display to a user.

13. The system of claim 12, further including receiving information relating to an age, ethnicity, ancillary test results, and/or an autopsy report of the patient.

14. The system of claim 12, further including detecting one or more salient region of each of the plurality of electronic medical images.

15. The system of claim 14, where the machine learning system only analyzes the salient regions of the plurality of electronic medical images.

16. The system of claim 12, wherein the machine learning system determines a numerical value score for each contributing cause of death.

17. The system of claim 12, further including marking the plurality of medical images to determine where evidence for the contributing cause of death is located.

18. The system of claim 12, wherein when more than one contributing cause of death is determined, ranking each contributing cause of death from most to least likely.

19. The system of claim 12, including determining and ranking which of the plurality of medical images provides the most evidence for the contributing cause of death.

20. A non-transitory computer-readable medium storing instructions that, when executed by a processor, perform operations processing electronic medical images, the operations comprising:
receiving a plurality of electronic medical images of at least one pathology specimen, the pathology specimen being associated with a patient;
determining, using a machine learning system and based on the electronic medical images, at least on contributing cause of death, wherein the machine learning system is trained using a plurality of electronic medical images; and
providing the at least one contributing cause of a death for display to a user.
